(19) United States
(12) Patent Application Publication
(10) Pub. No.: US 2014/0207004 A1
(43) Pub. Date: Jul. 24, 2014

(54) SYSTEM AND METHOD FOR DETERMINING RESPIRATORY EFFORT

(71) Applicant: COVIDIEN LP, Boulder, CO (US)

(72) Inventors: Paul Stanley Addison, Edinburgh (GB); James Nicholas Watson, Dunfermline (GB)

(73) Assignee: Covidien LP, Boulder, CO (US)

(21) Appl. No.: 13/744,666

(22) Filed: Jan. 18, 2013

(51) Int. Cl. A61B 5/00; A61B 7/02; A61B 8/00; A61B 5/0476; A61B 5/0488; A61B 5/0205; A61B 5/0402

(52) U.S. Cl. CPC A61B 5/7278; A61B 5/0205; A61B 5/7225; A61B 5/6852; A61B 5/0004; A61B 5/742; A61B 7/02; A61B 5/7475; A61B 5/0402; A61B 5/0476; A61B 5/0488; A61B 8/00; A61B 5/746
USPC 600/484

(57) ABSTRACT

A system for determining respiratory effort of an individual may include a pressure signal determination module configured to determine a physiological pressure signal of the individual, a wavelet transform module configured to transform the physiological pressure signal into a scalogram using at least one wavelet transform, and a respiratory effort determination module configured to determine the respiratory effort of the individual through an analysis of scalogram.

### FIELD

Embodiments of the present disclosure generally relate to physiological signal processing and, more particularly, to a system and method that uses one or more wavelet scalograms of a physiological pressure signal, such as a blood pressure signal, to determine respiratory effort.

### BACKGROUND

Blood pressure represents a measurement that quantifies a pressure exerted by circulating blood upon walls of blood vessels. In general, blood pressure is an example of a principal vital sign. Typically, blood pressure may be measured through use of a sphygmomanometer, or blood pressure cuff, and a stethoscope. However, blood pressure may also be invasively detected through an arterial line catheter, for example.

Additionally, blood pressure may be measured indirectly through analysis of a photoplethysmography (PPG) signal. PPG is a non-invasive, optical measurement that may be used to detect changes in blood volume within tissue, such as skin, of an individual. PPG may be used with pulse oximeters, vascular diagnostics, and digital blood pressure detection systems. Typically, a PPG system includes a light source that is used to illuminate tissue of a patient. A photodetector is then used to measure small variations in light intensity associated with blood volume changes proximal to the illuminated tissue. However, determination of blood pressure through a PPG signal may not always be completely accurate, as the determination is typically an indirect measurement, as opposed to a direct measure of the blood pressure itself.

### SUMMARY

Certain embodiments of the present disclosure provide a system for determining respiratory effort of an individual. The system may include a pressure signal determination module configured to determine a physiological pressure signal of the individual, a wavelet transform module configured to transform the physiological pressure signal into a scalogram using at least one wavelet transform, and a respiratory effort determination module configured to determine the respiratory effort of the individual through an analysis of the scalogram.

The system may also include a pressure detection sub-system configured to directly detect a physiological pressure of the individual. The pressure signal determination module determines the physiological pressure signal from the directly-detected physiological pressure of the individual. The pressure signal determination module may be operatively connected to the pressure detection sub-system. The pressure signal determination module may be configured to receive the physiological pressure from the pressure detection sub-system. The pressure detection sub-system may include a pressure detection device having an arterial line (A-line) catheter configured to be implanted within vasculature of the individual. The A-line catheter is configured to directly detect the physiological pressure within the vasculature of the patient.

The scalogram decouples the physiological pressure signal into multiple component parts. The respiratory effort determination module is configured to determine the respiratory effort of the individual through an analysis of one or more of the multiple component parts. In an embodiment, the scalogram includes one or more of a primary respiratory band, a secondary respiratory band, a primary pulse band, or a secondary pulse band. The respiratory effort determination module may be configured to determine the respiratory effort of the individual through an analysis of one or more of the primary respiratory band, the second respiratory band, the primary pulse band, or the secondary pulse band.

The respiratory effort determination module may be configured to analyze the scalogram to determine a ridge amplitude defined by a signal amplitude multiplied by a constant. In an embodiment, the constant is 0.67.

Certain embodiments of the present disclosure provide a method for determining respiratory effort of an individual. The method may include determining a physiological pressure signal of the individual with a pressure signal determination module, transforming the physiological pressure signal into a scalogram through at least one wavelet transform using a wavelet transform module, and determining the respiratory effort of the individual through an analysis of the scalogram using a respiratory effort determination module.

Certain embodiments of the present disclosure provide a tangible and non-transitory computer readable medium that includes one or more sets of instructions configured to direct a computer to determine a physiological pressure signal of the individual through a direct detection of the physiological pressure, transform the physiological pressure signal into a scalogram through at least one wavelet transform, and determine the respiratory effort of the individual through an analysis of the scalogram.

### BRIEF DESCRIPTION OF THE DRAWINGS

FIG. 1 illustrates a simplified block diagram of a system for determining respiratory effort, according to an embodiment of the present disclosure.

FIG. 2 illustrates a simplified block diagram of a pressure detection sub-system, according to an embodiment of the present disclosure.

FIG. 3 illustrates a simplified front view of a pressure detection sub-system, according to an embodiment of the present disclosure.

FIG. 4 illustrates a simplified lateral view of a pressure detection device, according to an embodiment of the present disclosure.

FIG. 5 illustrates a blood pressure signal over time, according to an embodiment of the present disclosure.

FIG. 6(a) illustrates a top plan view of a scalogram derived from a pressure signal, according to an embodiment of the present disclosure.

FIG. 6(b) illustrates an isometric top view of a scalogram derived from a pressure signal, according to an embodiment of the present disclosure.

FIG. 6(c) illustrates an exemplary scalogram derived from a signal containing two pertinent components, according to an embodiment of the present disclosure.

FIG. 6(d) illustrates a schematic of signals associated with a ridge in FIG. 6(c), and a schematic of a further wavelet decomposition of the signals, according to an embodiment of the present disclosure.

FIG. 7 illustrates a signal over time, according to an embodiment of the present disclosure.

FIG. 8 illustrates an isometric top view of a scalogram, according to an embodiment of the present disclosure.

FIG. 9 illustrates a two-dimensional plot of a scalogram with respect to characteristic frequency and amplitude, according to an embodiment of the present disclosure.

FIG. 10 illustrates a physiological pressure signal over time, according to an embodiment of the present disclosure.

FIG. 11 illustrates an isometric top view of a scalogram derived from a physiological pressure signal, according to an embodiment of the present disclosure.

FIG. 12 illustrates an amplitude of a physiological pressure signal over time extracted from a scalogram surface, according to an embodiment of the present disclosure.

FIG. 13 illustrates a characteristic frequency of a physiological pressure signal over time extracted from a scalogram surface, according to an embodiment of the present disclosure.

FIG. 14 illustrates a scalogram of a physiological pressure signal over time, according to an embodiment of the present disclosure.

FIG. 15 illustrates a method of determining respiratory effort, according to an embodiment of the present disclosure.

### DETAILED DESCRIPTION

FIG. 1 illustrates a simplified block diagram of a system 100 for determining respiratory effort, according to an embodiment of the present disclosure. The system 100 may include a pressure signal determination module 102, a wavelet transform module 104, and a respiratory effort determination module 106. The system 100 is configured to determine respiratory effort of an individual based on a physiological pressure signal, such as a blood pressure signal. The pressure signal determination module 102, the wavelet transform module 104, and the respiratory effort determination module 106 may be connected to one another through cables, wireless connections, and/or the like.

The pressure signal determination module 102 may be configured to receive a physiological pressure, such as blood pressure, from a pressure detection device (not shown in FIG. 1) that is configured to detect the physiological pressure of an individual. The pressure signal determination module 102 may analyze the physiological pressure and display a pressure signal based on the physiological pressure as a two-dimensional waveform.

The wavelet transform module 104 receives the pressure signal, such as a pressure waveform, from the pressure signal determination module 102. The wavelet transform module 104 is configured to transform the pressure signal with one or more wavelet transforms to yield a scalogram, such as a rescaled wavelet scalogram.

The respiratory effort determination module 106 is configured to analyze the wavelet scalogram to determine respiratory effort. As such, a direct measurement of blood pressure, such as through an arterial line (A-line) catheter may be used to determine respiratory effort, as described in further detail below. Embodiments of the present disclosure provide a system and method in which direct measures of respiratory effort may be extracted from the wavelet scalogram of a physiological pressure trace, such as a blood pressure trace.

The system 100 may be contained within a workstation that may be or otherwise include one or more computing devices, such as standard computer hardware. Each module 102, 104, and 106 may include one or more control units, such as processing devices that may include one or more microprocessors, microcontrollers, integrated circuits, memory, such as read-only and/or random access memory, and the like.

The modules 102, 104, and 106 may be integrated and contained within a single housing. Alternatively, each module 102, 104, and 106 may be contained within a respective housing.

The system 100 may also include a display 108, such as a cathode ray tube display, a flat panel display, such as a liquid crystal display (LCD), light-emitting diode (LED) display, a plasma display, or any other type of monitor. The system 100 may be configured to calculate physiological parameters and to show information related to pressure, such as blood pressure, and respiratory effort of a patient on the display 108.

The system 100 may include any suitable computer-readable media used for data storage. For example, one or more of the modules 102, 104, and 106 may include computer-readable media. The computer-readable media are configured to store information that may be interpreted by the modules 102, 104, and 106. The information may be data or may take the form of computer-executable instructions, such as software applications, that cause a microprocessor or other such control unit within the modules 102, 104, and 106 to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

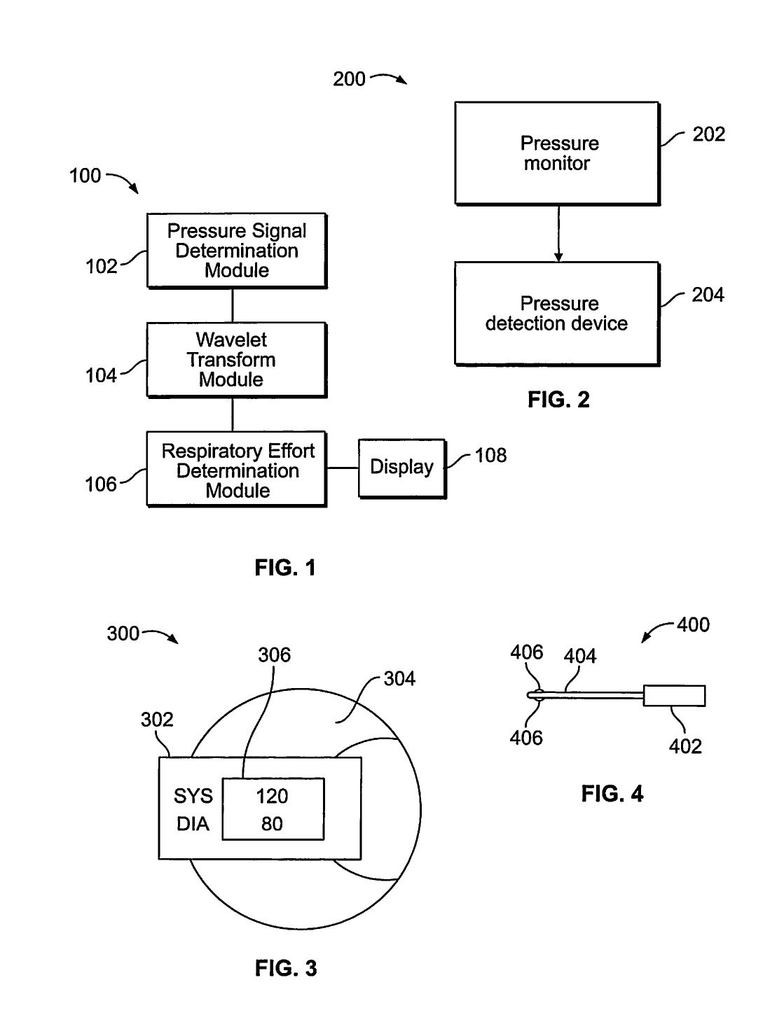

FIG. 2 illustrates a simplified block diagram of a pressure detection sub-system 200, according to an embodiment of the present disclosure. The pressure detection sub-system 200 may be configured to detect blood pressure of an individual, and may include a pressure monitor 202, such as a blood pressure monitor, operatively connected to a pressure detection device 204, such as a blood pressure detection device. The pressure detection device 204 may be configured to be secured to a portion of a patient. For example, the pressure detection device 204 may be a blood pressure cuff configured to be removably secured around a portion of patient anatomy, such as an arm. Optionally, the pressure detection device 204 may be configured to be implanted within patient anatomy, such as within a portion of an artery. For example, the pressure detection device 204 may include an A-line catheter.

The pressure monitor 202 is operatively connected to the pressure detection device 204, such as through mechanical and/or electrical connections. For example, the pressure monitor 202 may be connected to the pressure detection device 204 through cables, wires, and/or wireless connections. The pressure monitor 202 receives physiological pressure measurements from the pressure detection device 204. In this manner, the pressure sub-system 200 directly detects pressure, such as blood pressure, of an individual. The pressure sub-system 200 may be in communication with the pressure signal determination module 102 (shown in FIG. 1), such as through cables, wires, and/or wireless connections. The pressure signal determination module 102 may receive the detected pressure from the pressure detection device 204, and analyzes the detected pressure to determine the pressure, such as the blood pressure, of the individual.

FIG. 3 illustrates a simplified front view of a pressure detection sub-system 300, according to an embodiment of the present disclosure. The pressure detection sub-system 300 may be a blood pressure detection sub-system that includes a blood pressure monitor 302 operatively connected to a blood pressure cuff 304 configured to be positioned around a portion of an arm of an individual. As shown in FIG. 3, the blood pressure sub-system 300 may allow for non-invasive blood pressure detection by way of the cuff 304 being positioned around a portion of patient anatomy. The blood pressure monitor 302 may include a digital blood pressure monitor having a display 306 that shows blood pressure data. The blood pressure monitor 302 may be in communication with the pressure signal determination module 102 (shown in FIG. 1) of the system 100. Alternatively, the blood pressure monitor 302 may include the pressure signal determination module 102. Also, alternatively, the blood pressure sub-system 300 may include a sphygmomanometer and stethoscope used by an individual to detect blood pressure. A direct measure of physiological pressure, such as blood pressure, may be input into the pressure signal determination module 102, which may be in communication with the pressure detection sub-system 300. Alternatively, the pressure detection sub-system 300 may not be in direction communication with the pressure signal determination module 102. In this embodiment, blood pressure data may be presented on the display 306, and an individual, such as hospital personnel, may then input the data into the system 100 (shown in FIG. 1), such as through a keyboard, mouse, or the like, and analyzed by the pressure signal determination module 102.

FIG. 4 illustrates a simplified lateral view of a pressure detection device 400, according to an embodiment of the present disclosure. The pressure detection device 400 may be a blood pressure detection device that includes a housing 402 connected to a catheter 404 configured to be positioned within vasculature of a patient. The catheter 404 may include one or more pressure detection sensors 406, such as piezoelectric transducers, at various points along its length. The pressure detection sensors 406 are configured to detect pressure pulses of blood within the vasculature. The blood pressure detection device 400 may include, for example, an A-line catheter. The blood pressure detection device 400 may be operatively connected to and in communication with the blood pressure monitor 202 (shown in FIG. 2).

The pressure detection device 400 may include a thin catheter configured to be inserted into an artery of a patient. Accordingly, the blood pressure detection device 400 may be used to directly detect blood pressure in real time, rather than through intermittent measurement, and/or through analysis of another physiological signal. The blood pressure detection device 400 may be inserted into the radial artery proximate the wrist, the brachial artery proximate the elbow, the femoral artery proximate the groin, the dorsalis pedis artery proximate the foot, or into the ulnar artery inside the wrist, for example. However, the blood pressure detection device 400 may be configured to be positioned within various other arteries, veins, and vasculature at various other portions of a patient's body.

Referring to FIGS. 2-4, the pressure detection sub-system 200 may be any type of system configured to detect a physiological pressure, such as blood pressure. FIGS. 3 and 4 merely provide examples of such a sub-system 200, pressure monitor 202, and pressure detection device 204. The pressure detection device 204 may be an invasive, non-invasive, or minimally invasive device configured to detect blood pressure. For example, the blood pressure detection device 204 may be various types of invasive, non-invasive tonometric and volume clamping systems, as well as auscultation, oscillometric and other such devices.

The pressure monitor 202 may be configured to calculate a physiological pressure, such as blood pressure, of an individual based at least in part on pressure pulses received from the pressure detection device 204. The pressure monitor 202 may include a display, such as the display 306, configured to display blood pressure. The pressure detection device 204 may be communicatively coupled to the pressure monitor 202 via a cable, wireless connection, or the like. The pressure monitor 202 may include one or more modules and control units, such as processing devices that may include one or more microprocessors, microcontrollers, integrated circuits, memory, such as read-only and/or random access memory, and the like. Accordingly, the pressure monitor 202 may be configured to calculate a physiological pressure and to show information related to the physiological pressure on a display. The pressure monitor 202 may be communicatively coupled to the system 100 (shown in FIG. 1) via a cable that is coupled to a sensor input port or a digital communications port, respectively and/or may communicate wirelessly with the system 100. Additionally, the pressure monitor 202 may be coupled to a network to enable the sharing of information with servers or other workstations. The pressure monitor 202 may be powered by a battery or by a conventional power source such as a wall outlet.

The pressure detection sub-system 200 may be configured to detect the pressure exerted by circulating blood within vasculature of an individual. During each heartbeat, blood pressure varies between a maximum (systolic) and a minimum (diastolic) pressure.

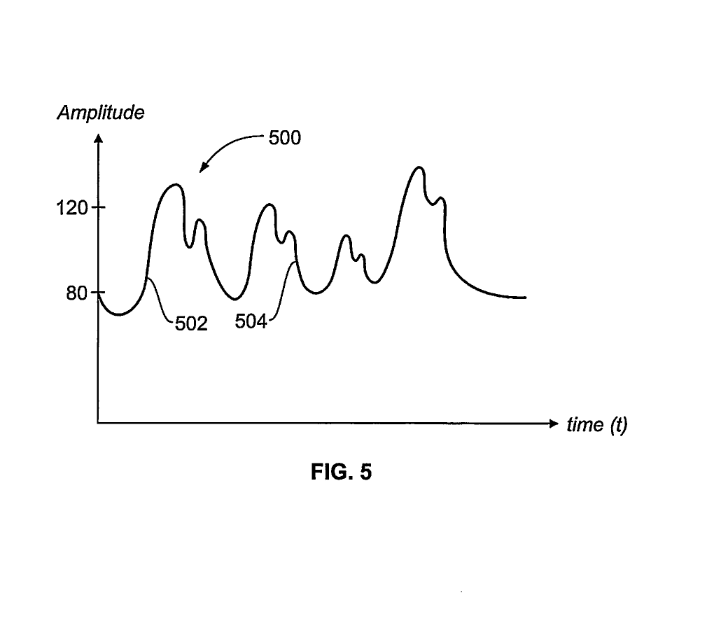

FIG. 5 illustrates a blood pressure signal 500 over time, according to an embodiment of the present disclosure. The blood pressure signal 500 is an example of a physiological signal. As shown, a physiological parameter, such as an amplitude of the blood pressure signal 500, may vary over time. For example, the amplitude may vary with respect to a base, average, or mean blood pressure of 120 systolic over 80 diastolic. As an example, the amplitude may change from blood pressure pulse 502 to blood pressure pulse 504. The pressure signal determination module 102 (shown in FIG. 1) and/or the pressure detection sub-system 200 (shown in FIG. 2), may track the change in amplitude of the blood pressure signal 500, and store the change in amplitude for analysis. The pressure signal determination module 102 and/or the pressure detection sub-system 200 may track and store amplitude changes between neighboring blood pressure pulses, such as pulses 202 and 204. Alternatively, the pressure signal determination module 102 may determine an average amplitude modulation over a particular time frame, for example. Also, alternatively, the pressure signal determination module 102 may determine an amplitude change of a blood pressure signal by directly comparing blood pressure waveforms. For example, a first blood pressure waveform may be superimposed over a second blood pressure waveform, and the pressure signal determination module 102 may determine a change between blood pressure waveforms through the difference in waveform shapes. For example, an amplitude change between the first and second blood pressure waveforms may be a difference between a maximum and a minimum amplitude over a respiratory cycle. The amplitude change may represent an average of the difference between the maximum and minimum amplitudes of the blood pressure waveforms over a number of respiratory cycles.

Referring again to FIGS. 1 and 2, the system 100 is configured to directly determine a physiological pressure signal from a physiological pressure that is detected through a pressure detection sub-system 200. As described above, the physiological pressure signal may be a blood pressure signal of an individual. However, the system 100 may be configured to detect various other physiological pressure signals. For example, the system 100 may be used to determine a range of blood pressure traces, such as through an A-line, a continuous non-invasive blood pressure signal, pleural pressure, central venous pressure, esophageal pressure, and the like.

The pressure signal determination module 102 receives a physiological pressure, such as blood pressure, from the pressure detection sub-system 200. In particular, the physiological pressure is detected with the pressure detection device 204. The pressure monitor 202 may determine the physiological pressure. The pressure signal determination module 102 may receive the physiological pressure from the pressure monitor 202 and analyzes and displays a physiological pressure signal based on the physiological pressure as a two-dimensional waveform, such as shown in FIG. 5.

The wavelet transform module 104 receives the pressure waveform from the pressure signal determination module 102. The wavelet transform module 104 transforms the pressure waveform with one or more wavelet transforms to yield a scalogram, such as a rescaled pressure scalogram.

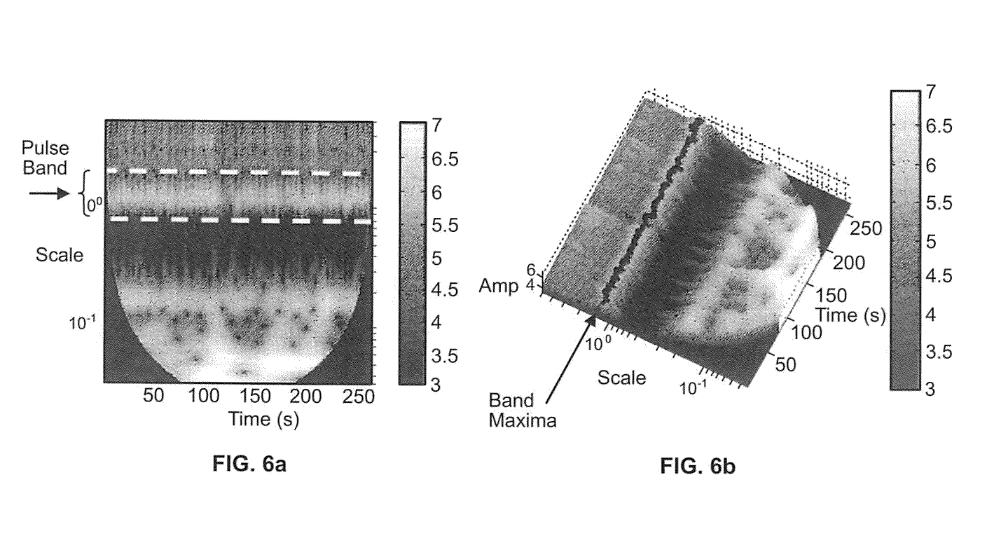

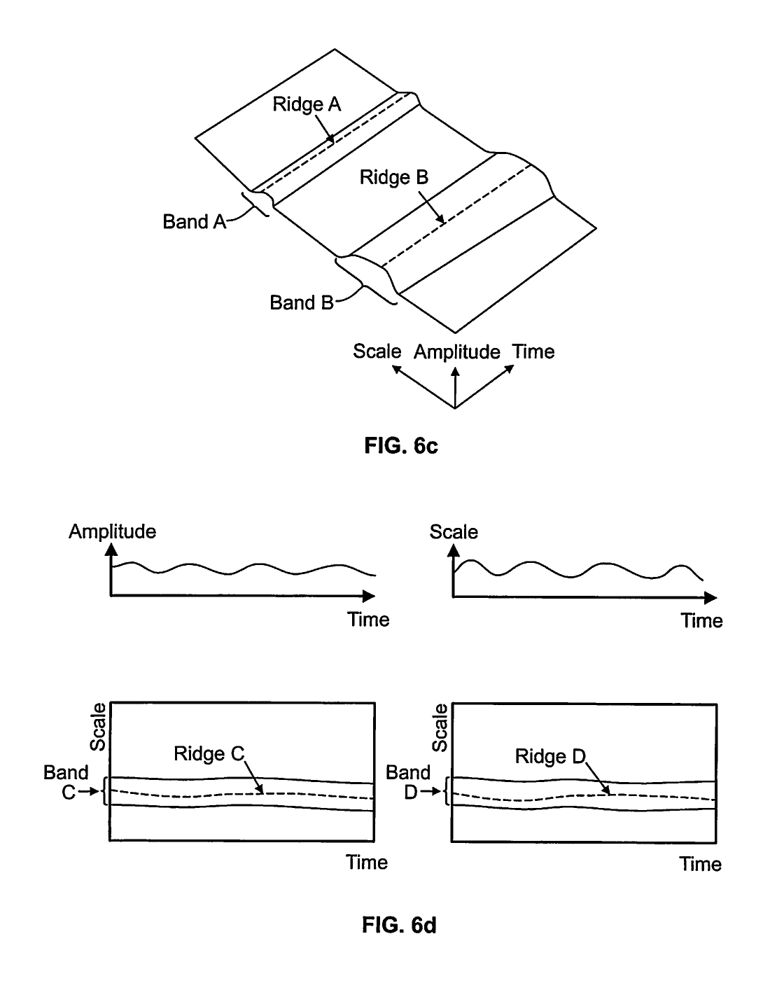

FIGS. 6(a) and 6(b) illustrate top plan and isometric views, respectively, of a scalogram derived from a pressure signal, according to an embodiment of the present disclosure. A pressure signal, such as the blood pressure signal 500 shown in FIG. 5, may be transformed using a continuous wavelet transform. Information derived from the transform of the pressure signal may be used to provide measurements of one or more physiological parameters.

The wavelet transform of a signal x(t) may be defined as shown in Equation (1):

$$T ( a , b ) = \frac{1}{\sqrt{a}} {\int }_{- ∞}^{+ ∞} x ( t ) {ψ}^{*} ( \frac{t - b}{a} ) d t \qquad Equation ( 1 )$$

where ψ*(t) is the complex conjugate of the wavelet function ψ(t), a is the dilation or scale parameter of the wavelet, b is the location parameter of the wavelet and x(t) is the signal under investigation. For example, x(t) may be a physiological pressure signal, such as a blood pressure signal or waveform, as shown in FIG. 5.

The transform given by Equation (1) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The wavelet transform decomposes a signal using wavelets, which are generally highly localized in time. A continuous wavelet transform may provide a higher resolution relative to discrete transforms, thus providing the ability to garner more information from signals that typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain create a series of frequency transform values in a two-dimensional coordinate system where the two dimensions may be frequency and, for example, amplitude. Wavelet transforms are further described in U.S. Pat. No. 7,944,551, entitled “Systems and Methods for a Wavelet Transform Viewer,” and U.S. Patent Application Publication No. 2010/0079279, entitled “Detecting a Signal Quality Decrease in a Measurement System,” both of which are hereby incorporated by reference in their entireties.

Referring again to Equation (1), a modulus of the transform may be defined as |T(a,b)|. As such, the energy density function of the wavelet transform (that is, the scalogram) may be rescaled as follows:

$${\vert T ( a , b ) \vert}^{*} = \frac{\vert T ( a , b ) \vert}{\sqrt{a}} \qquad Equation ( 2 )$$

The rescaled wavelet transform scalogram may be used to define ridges in wavelet space, such as when a Morlet wavelet is used. However, any suitable wavelet function may be used with embodiments of the present disclosure. The scalogram may be taken to include all suitable forms of rescaling including, but not limited to, the original unscaled wavelet representation, linear rescaling, any power of the modulus of the wavelet transform, or any other suitable rescaling. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”. A ridge is a locus of points of local maxima in a plane. Also, a ridge may be a path displaced from the locus of the local maxima. The rescaled wavelet transform scalogram allows for a representation in which ridges of bands on the transform surface scale directly with amplitudes of corresponding signal components. By using the rescaled transform, the direct scale relationship may take the simplified form as follows:

Aᵣ=K·Aₛ Equation (3)

where Aᵣ is the ridge amplitude, K is a constant, and Aₛ is the signal amplitude, which may be defined as the distance from peak-to-trough. For a sinusoidal signal, Equation (3) may take the form of the following:

$${A}_{r} = \frac{\sqrt[4]{π}}{2} {A}_{s} \qquad Equation ( 4 )$$

Accordingly, the ridge amplitude may be related to the signal amplitude by a constant (K) of 0.67. That is, K=\sqrt{π}/2. However, other constants may be experimentally or empirically derived, based on various factors, such as the type of wavelet transform being used.

Wavelet transform features may be extracted from the wavelet decomposition of signals. For example, wavelet decomposition of physiological pressure signals, such as blood pressure signals, may be used to provide clinically useful information.

Pertinent repeating features in a signal give rise to a time-scale band in wavelet space or a resealed wavelet space. For example, the pulse component of a physiological pressure signal produces a dominant band in wavelet space at or around the pulse frequency. FIGS. 6(a) and 6 (b) illustrate two views of an illustrative scalogram derived from a blood pressure signal, according to an embodiment of the present disclosure. The figures show an example of the band caused by the pulse component in such a signal. The pulse band is located between the dashed lines in the plot of FIG. 6(a). The pulse band is formed from a series of dominant coalescing features across the scalogram. The pulse band is more clearly seen as a raised band across the transform surface in FIG. 6(b) located within the region of scales indicated by the arrow in the plot. The maxima of the pulse band with respect to the scale is the ridge. The locus of the ridge is shown as a black curve on top of the band in FIG. 6(b). By employing a suitable rescaling of the scalogram, such as that given in equation (2), the ridges found in wavelet space may be related to the instantaneous frequency of the signal. In this way, the pulse rate may be obtained from the signal. Instead of rescaling the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. As such, both times between individual pulses and the timing of components within each pulse may be monitored and used to detect heart beat anomalies, measure arterial system compliance, or perform any other suitable calculations or diagnostics.

FIG. 6(c) illustrates an exemplary scalogram derived from a signal containing two pertinent components, according to an embodiment of the present disclosure. As noted above, pertinent repeating features in the signal give rise to a time-scale band in wavelet space or a rescaled wavelet space. For a periodic signal, the band remains at a constant scale in the time-scale plane. For many real signals, especially biological signals, the band may be non-stationary—varying in scale, amplitude, or both over time. As shown in FIG. 6(c), the two pertinent components lead to two bands in the transform space. The bands are labeled band A and band B on the three-dimensional schematic of the wavelet surface. In an embodiment, the band ridge is defined as the locus of the peak values of the bands with respect to scale. For purposes of clarity, it may be assumed that band B contains the signal information of interest. As such, band B may be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B, then the information within band B can become ambiguous (for example, obscured, fragmented, or missing). As such, the ridge of band A may be followed in wavelet space and extracted either as an amplitude signal or a scale signal which may be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively.

FIG. 6(d) illustrates a schematic of signals associated with a ridge in FIG. 6(c), and a schematic of a further wavelet decomposition of the signals, according to an embodiment of the present disclosure. The top plots of FIG. 6(d) illustrate a schematic of the RAP and RSP signals associated with ridge A in FIG. 6(c). Below the RAP and RSP signals are schematics of a further wavelet decomposition of the newly derived signals. The secondary wavelet decomposition allows for information in the region of band B in FIG. 6(c) to be made available as band C and band D. The ridges of bands C and D may serve as instantaneous time-scale characteristic measures of the signal components causing bands C and D. This technique, which may be referred to as secondary wavelet feature decoupling (SWFD), may allow information concerning the nature of the signal components associated with the underlying physical process causing the primary band B (shown in FIG. 6(c)) to be extracted when band B itself is obscured in the presence of noise or other erroneous signal features.

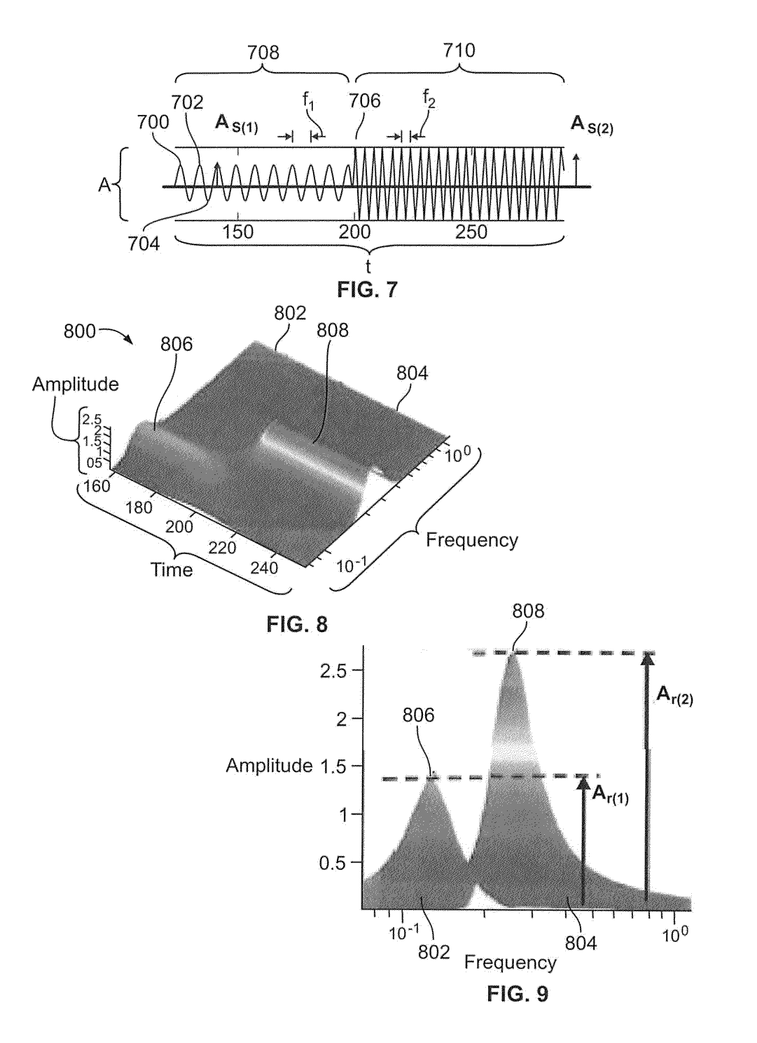

FIG. 7 illustrates a signal 700 over time, according to an embodiment of the present disclosure. The signal 700 is plotted over time t, and has an amplitude A. The signal 700 may be a generic test sinusoidal signal. As such, the signal 700 may not include various components found in an actual physiological pressure signal. As shown in FIG. 7, the amplitude A of the signal is measured from a peak 702 to a trough 704 of the signal 700. The amplitude A of the signal changes at a midpoint 706 of the signal trace. In the first half 708 of the signal trace, the amplitude A_{S(1)} is approximately half the amplitude A_{S(2)} of the signal in the second half 710 of the signal trace. Further, a frequency f₁ of the signal 700 in the first half 708 of the signal trace may be less than the frequency f₂ of the signal 700 in the second half 710 of the signal trace.

In general, the signal 700 may be determined by the pressure signal determination module 102 (shown in FIG. 1). The pressure signal determination module 102 may present the pressure signal 700 on a display of the system 100. After the system 100 determines the pressure signal 700, the wavelet transform module 104 (shown in FIG. 1) uses one or more wavelet transforms to transform the pressure signal 700 into a scalogram, as described above.

FIG. 8 illustrates an isometric top view of a scalogram 800, according to an embodiment of the present disclosure. The scalogram 800 represents the pressure signal 700 after it has been transformed by the wavelet transform module 104 (shown in FIG. 1). The wavelet transform module 104 uses a wavelet transform to decouple components of the pressure signal 700 (shown in FIG. 1). The scalogram 800 is plotted with respect to characteristic frequency, time, and amplitude. The scalogram 800 includes a first band 802 correlated with the first half of the signal trace shown in FIG. 7, and a second band 804 correlated with the second half of the signal trace shown in FIG. 7. The first band 802 includes a first ridge 806, while the second band 804 includes a second ridge 808.

FIG. 9 illustrates a two-dimensional plot of the scalogram 800 with respect to characteristic frequency and amplitude, according to an embodiment of the present disclosure. As shown in FIG. 9, the amplitude Aᵣ₍₁₎ of the ridge 806 of the first band 802 is less than the amplitude Aᵣ₍₂₎ of the ridge 808 of the second band 808.

Referring to FIGS. 8 and 9, the scalogram 800 represents a rescaled wavelet transform scalogram corresponding to the pressure signal 700 shown in FIG. 7. The magnitudes of the ridges 806 and 808 correspond to the first half 708 and the second half 710, respectively, of the pressure signal 700. Notably, the change in signal frequency does not affect the amplitude of the ridges 806 and 808 in the scalogram 800. Accordingly, a measure of the signal component amplitude may be discerned, detected, or otherwise observed from the surface of the scalogram 800. While the pressure signal 700 shown in FIG. 7 is a simplified sinusoidal signal, the described system and method for determining ridge amplitudes is particularly useful for more complex signals, such as blood pressure signals. By transforming the pressure signal 700 into the scalogram 800, the wavelet transform module 104 (shown in FIG. 1) allows various signal components of the pressure signal 700 to be decoupled. Because signal components are decoupled from one another, the respiratory effort determination module 106 (shown in FIG. 1) may analyze the decoupled signal components to determine the respiratory effort of an individual. That is, the system 100 determines a physiological pressure signal, such as a blood pressure signal, and transforms the physiological pressure signal into a scalogram, which decouples signal components of the physiological pressure signal from one another. The system 100 is then able to clearly and unambiguously analyze the decoupled signal components to determine various physiological characteristics associated with the respective decoupled signal components. For example, the respiratory effort determination module 106 is able to determine respiratory effort from at least one signal component, such as one or more respiratory bands, that is/are defined in the scalogram 800.

As explained below, the system and method described above may be applied to a complex physiological signal.

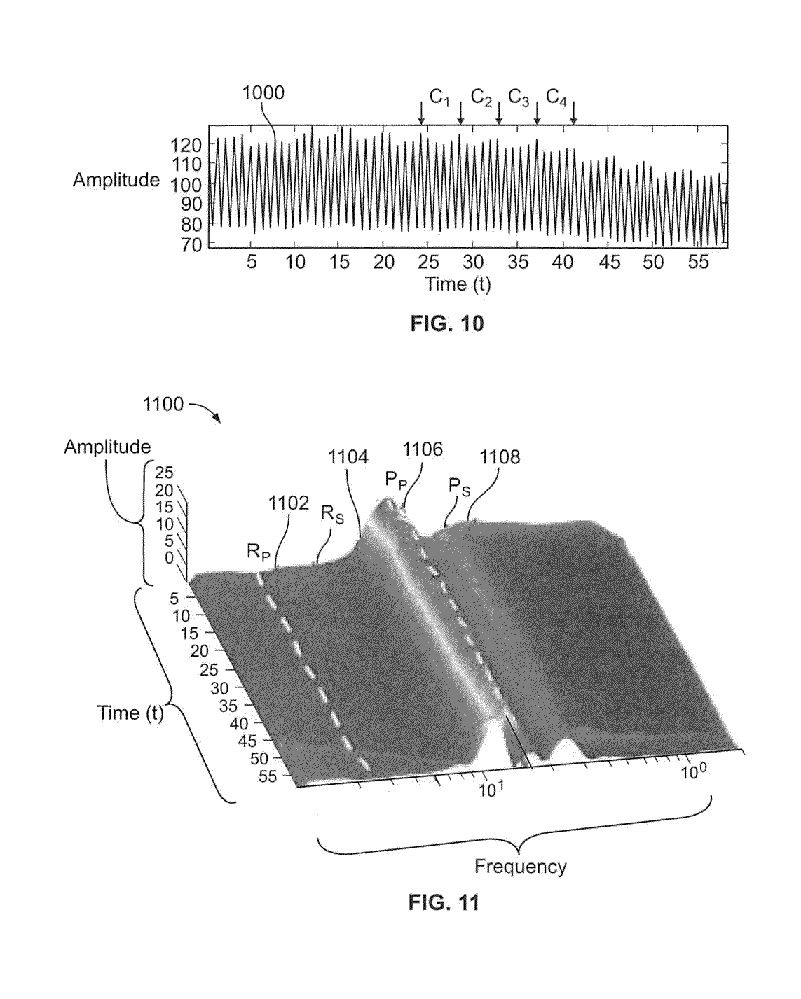

FIG. 10 illustrates a physiological pressure signal 1000 over time t, according to an embodiment of the present disclosure. The pressure signal 1000 may be a blood pressure signal detected by a blood pressure detection sub-system, such as the sub-system 200 shown in FIG. 2. The blood pressure signal 1000 may be detected through an A-line catheter, for example. The pressure signal 1000 is received by the pressure signal determination module 102 (shown in FIG. 1), which analyzes the amplitude of the pressure signal 1000 over time t. Four respiration cycles C₁, C₂, C₃, and C₄ are noted in the physiological pressure signal 1000.

FIG. 11 illustrates an isometric top view of a scalogram 1100 derived from the physiological pressure signal 1000 shown in FIG. 10, according to an embodiment of the present disclosure. As explained above, the wavelet transform module 104 (shown in FIG. 1) transforms the physiological pressure signal 1000 into the scalogram 1100. The scalogram 1100 may include one or more respiration bands and one or more pulse bands. Each of the respiration and pulse bands may include primary and secondary signals, which may be characteristic of particular morphology of the pressure signal 1000. Cycles C₁-C₄ depicted in FIG. 10 represent respiration modulations superimposed on a cardiac pulse signal. The respiratory modulations produce a primary respiration band 1102 having a ridge Rₚ, and a secondary respiration band 1104 having a ridge Rₛ. The cardiac pulse signal produces a primary pulse band 1106 having a ridge Pₚ, and a secondary pulse band 1108 having a ridge Pₛ. The scalogram 1100 decouples or otherwise separates the various bands 1102, 1104, 1106, and 1108 from the physiological pressure signal 1000 (shown in FIG. 10). The respiratory effort determination module 106 (shown in FIG. 1) may analyze each of the bands 1102, 1104, 1106, and 1108 to determine various physiological parameters. For example, the respiratory effort determination module 106 may analyze one or both of the primary and secondary respiration bands 1102 and 1104, and/or one of both of the primary and secondary pulse bands 1106 and 1108 to determine respiratory effort. It is to be noted that the frequency axes shown in each of FIGS. 8, 9 and 11 refer to characteristic frequency.

As shown in FIG. 11, the respiration band, including the primary and secondary respiration bands 1102 and 1104, is smaller in magnitude than the pulse band, including the primary and secondary pulse bands 1106 and 1108. The respiration band is smaller than the pulse band because the respiration band represents baseline respiratory fluctuations that are generally an order of magnitude less than the pulse pressure. Using Equation (4) noted above, the amplitude of the respiration band may be converted to signal units. For example, Equation (4) may be used to convert the amplitude into units of mmHg, which may more directly correspond to pressure units commonly used with blood pressure readings. Analysis of the respiration band, including one or both of the primary and secondary respiration bands 1102 and 1104, may be correlated with an effort to breathe, or respiratory effort. Thus, the respiratory effort determination module 106 may analyze the respiration band, and utilize Equation (4) to determine the respiratory effort of an individual.

Referring to FIGS. 10 and 11, a portion of the physiological pressure signal 1000 may be used by the system 100 to ultimately determine respiratory effort. For example, more or less cycles may be used by the wavelet transform module 104 to convert the pressure signal 1000 into the scalogram 1100. In an embodiment, the wavelet transform module 104 may transform only the portion of the signal corresponding to cycles C₁ and C₂. In another embodiment, the wavelet transform module 104 may transform a sixty second trace of the physiological pressure signal 1000, which may then be analyzed by the respiratory effort determination module 106 to determine fluctuations of respiratory effort over the timeframe. However, greater or lesser timeframes may be used. Additionally, the respiratory effort determination module 106 may determine respiratory effort through analysis of the primary respiratory band 1102, the secondary respiratory band 1104, or both. Further, the respiratory effort determination module 106 may determine respiratory effort through analysis of the primary pulse band 1106, the secondary pulse band 1108, or both.

As shown in FIG. 11, the ridge Rₚ of the primary respiration band 1102 is plotted as a dashed line that is generally aligned with the time axis (t). The amplitude of the ridge Rₚ relates to a peak-to-trough amplitude of a pulsatile component in a pressure signal. For example, using Equation (4), if the amplitude of the ridge Rₚ is about 2.6, the pulsatile component of the amplitude is about 3.9 mmHg. In this manner, the respiratory effort determination module 106 may determine a pulsatile pressure signal component of the ridge Rₚ, and any other ridge of the scalogram, in terms of common pressure units.

In an embodiment, the respiratory effort determination module 106 may determine respiratory effort through an analysis of the primary respiration band 1102, such as through analysis of the ridge Rₚ. Alternatively, the secondary respiration band 1104 may be analyzed to determine respiratory effort. Further, the respiratory effort determination module 106 may analyze both the primary and secondary respiration bands 1102 and 1104 to determine respiratory effort. Additionally, the respiratory effort determination module 106 may analyze both the primary and secondary respiration bands 1102 and 1104 independent of one another to assess the accuracy of the determined respiratory effort.

Additionally, the respiratory effort determination module 106 may analyze the primary and secondary pulse bands 1106 and 1108 for respiratory modulations. The amplitude modulations of the pulse bands 1106 and 1008 may be indicative of a change in respiratory effort. As shown in FIG. 11, the ridge Pₚ of the primary pulse band 1106 is shown by a dashed line. Once again using Equation (4), the mean pulse ridge amplitude of the ridge Pₚ may correspond to a pulse pressure signal amplitude of a certain value measured in units mmHg. In general, there are distinct regular undulations in the pulse ridge amplitude, which relate to amplitude modulations of the pulse ridge caused by respiration. The undulations in the pulse ridge amplitude may, therefore, be analyzed by the respiratory effort determination module 106 and used as a primary, secondary, or backup determination of respiratory effort. As an example, a pulse ridge amplitude fluctuation of about 1.5 may correspond to about 2.2 mmHg fluctuation in the original pressure signal.

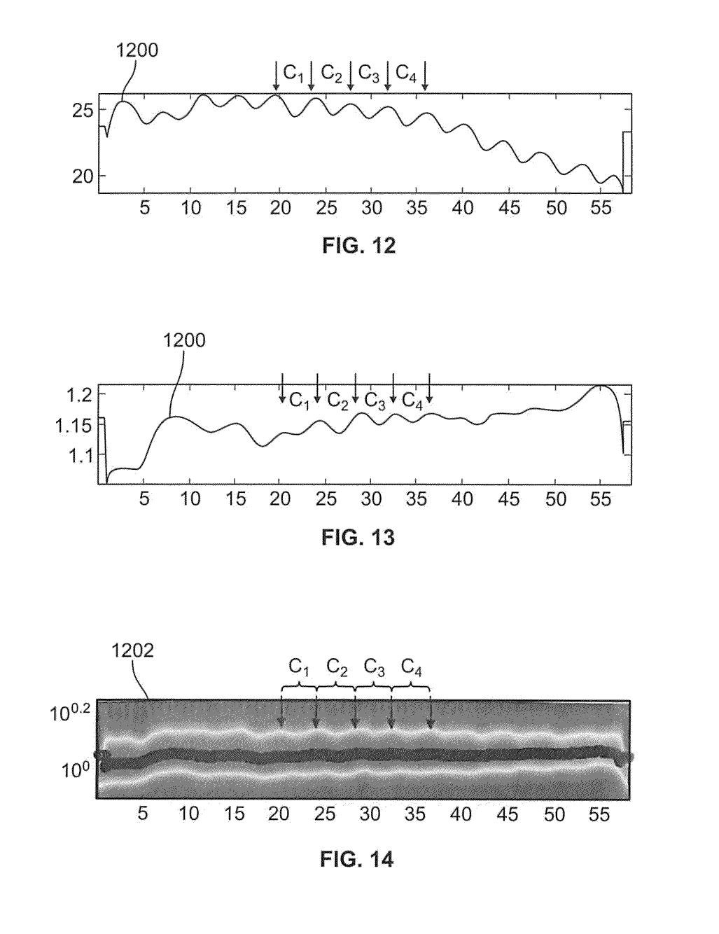

FIG. 12 illustrates a plot of pulse ridge amplitude of a pressure signal 1200 over time extracted from a scalogram surface, according to an embodiment of the present disclosure. FIG. 13 illustrates a plot of characteristic frequency of the physiological pressure signal 1200 over time extracted from the scalogram surface, according to an embodiment of the present disclosure. FIG. 14 illustrates the scalogram 1202 of the physiological pressure signal over time, according to an embodiment of the present disclosure. Referring to FIGS. 12-14, the respiratory cycles C₁, C₂, C₃, and C₄ are shown.

As shown in FIG. 12, a mean pulse ridge amplitude on the scalogram or transform surface is around 25, which corresponds to a pulse pressure signal amplitude of approximately 37 mmHg. As shown, there are distinct regular undulations in the pulse ridge amplitude. The undulations represent amplitude modulations of the pulse ridge caused by respiration. As shown, the pulse ridge amplitude fluctuation is around 1.5, which corresponds to about 2.2 mmHg fluctuations in the original pressure signal.

The pulse band amplitude modulations may be quantified by measuring the peak-to-trough values of the modulations directly from a plot, such as shown in FIG. 12, or they may be extracted using a second wavelet transform of the signal shown in FIG. 12.

Referring to FIG. 13, in particular, a respiratory sinus arrhythmia component of the scalogram may also be extracted from the transform surface. As shown in FIG. 13, the plan view of the pulse ridge is shown and may be seen to contain particular oscillations of the period of respiration. The oscillation component may change in amplitude or phase during periods of increased effort to breathe. The RSA measure may not be in mmHg, but rather as a difference in heart rate. However, the measure may be used in conjunction with any of the measurements described above to indicate changes in the effort to breathe. The RSA component may be used as a distinct identifier of a respiratory event, such as a sudden increase or decrease in an effort to breathe. The RSA component may be interpreted as a confidence or quality metric for the pressure values obtained using the systems and methods described above. The RSA component may also be used as a guide when selecting the area of the scalogram to search for features (for example, ridges) associated with breathing. Alternatively, other methods of respiratory rate measurement (for example, EtCO2) may be used to guide the selection of the search area for respiratory features in the scalogram.

In an embodiment, the measurement(s) of respiratory effort derived from the respiration band and the pulse band, as described above, may be used as separate indications of the effort to breathe, or they may be combined, such as through summation, average weighting, or the like, to yield a single measure of respiratory effort. Using Equation (4), the output(s) may be expressed in units of mmHg.

One or more of the respiratory effort measures described above may be monitored over time to detect long term changes in respiratory effort or isolated short term events of clinical significance.

Embodiments of the present disclosure provide a system and method of determining respiratory effort through a direct detection of physiological pressure, such as blood pressure, and determination of a physiological pressure signal derived from the physiological pressure. The embodiments may be used with various non-invasive and invasive systems and methods of detecting a physiological pressure signal.

Certain embodiments of the present disclosure may be based on the known relationship between a sinusoidal signal amplitude and its amplitude in wavelet space. However, certain components of a blood pressure signal may not be sinusoidal in nature, thereby causing secondary features in wavelet space to appear (for example, a secondary band). Therefore, the measured band amplitude may not represent the peak-to-trough amplitude of the component of interest of the pressure signal as energy from the signal is distributed to other parts of the transform surface. However, the derived signal component amplitude may be scaled by a modified constant to provide an improved measure of the respiratory effort parameter. In other words, the constant K, as shown in Equation (4), may be derived and/or chosen to be a different value than 0.67. The constant K may depend on the morphology of the pressure pulse and how its components are distributed in wavelet space.

Embodiments of the present disclosure provide a simple, yet robust method for deriving or otherwise determining respiratory effort for patients being monitored for a physiological pressure. Embodiments of the present disclosure provide a system and method that detects a physiological pressure, such as through an A-line, and directly transforms the pressure signal itself (as opposed to another signal, such as a PPG signal, from which pressure is indirectly determined) into a scalogram using one or more wavelet transforms. The scalogram decouples and separates component parts of the pressure signal from one another, and analyzes one or more of the component parts to determine a respiratory effort. Thus, respiratory effort may be determined through detection of a pressure signal.

Embodiments of the present disclosure provide a system and method for rapid indication of a change in the effort to breathe of the patient. Such an indication may trigger an alarm if it exceeds a threshold value. The threshold value may be an upper or lower value if, for example, a corresponding obstructive or central apnea type event occurs. In an embodiment, if respiratory effort exceeds a certain threshold, an alarm may be triggered. Similarly, if respiratory effort is below a certain threshold, an alarm may be triggered. Further, if a rate of change of respiratory effort over a predefined time exceeds a certain threshold, an alarm may be triggered.

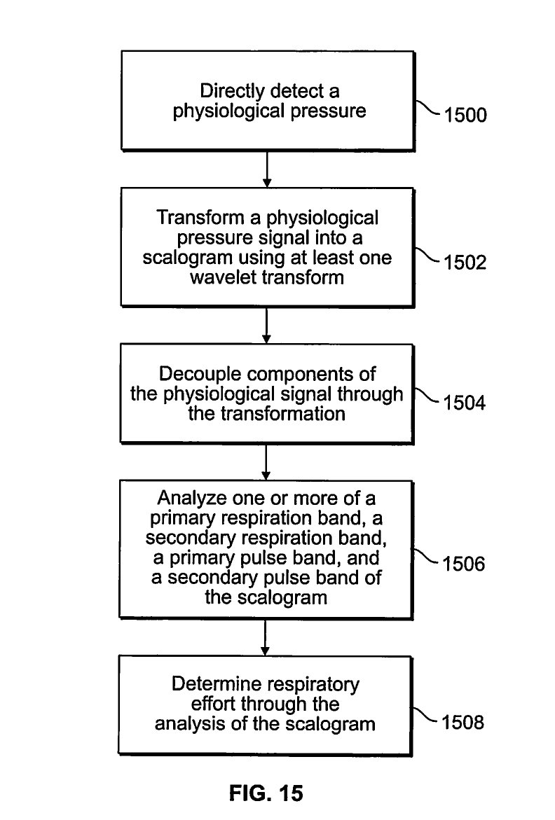

FIG. 15 illustrates a method of determining respiratory effort, according to an embodiment of the present disclosure. The method begins at 1500, in which a physiological pressure, such as blood pressure, is directly-detected. That is, the physiological pressure is detected through a physiological pressure detection sub-system, which is specifically configured to detect the physiological pressure (as opposed to the pressure signal being indirectly calculated through analysis of another signal, such as a PPG signal). Because the physiological pressure signal is directly-detected, a determination of the physiological pressure signal may be more accurate than if another physiological signal was used to estimate the physiological pressure signal.

Next, at 1502, a physiological pressure signal derived from the physiological pressure is transformed into a scalogram using at least one wavelet transform. At 1504, the components of the physiological pressure signal are decoupled or otherwise separated from one another through the transformation.

Then, at 1506, one or more of a primary respiration band, a secondary respiration band, a primary pulse band, and a secondary pulse band of the scalogram are analyzed. Respiratory effort is determined through the analysis of the scalogram at 1508.

It will be understood that the present disclosure may be applicable to any suitable physiological pressure signals and that blood pressure signals are used for illustrative purposes. Those skilled in the art will recognize that the present disclosure has wide applicability to other signals including, but not limited to other physiological signals (for example, electrocardiogram, electroencephalogram, electrogastrogram, electromyogram, heart rate signals, pathological sounds, ultrasound, or any other suitable biosignal) and/or any other suitable signal, and/or any combination thereof.

Various embodiments described herein provide a tangible and non-transitory (for example, not an electric signal) machine-readable medium or media having instructions recorded thereon for a processor or computer to operate a system to perform one or more embodiments of methods described herein. The medium or media may be any type of CD-ROM, DVD, floppy disk, hard disk, optical disk, flash RAM drive, or other type of computer-readable medium or a combination thereof.

The various embodiments and/or components, for example, the control units, modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor may also include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer” or “module.”

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

While various spatial and directional terms, such as top, bottom, lower, mid, lateral, horizontal, vertical, front, and the like may be used to describe embodiments, it is understood that such terms are merely used with respect to the orientations shown in the drawings. The orientations may be inverted, rotated, or otherwise changed, such that an upper portion is a lower portion, and vice versa, horizontal becomes vertical, and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A system for determining respiratory effort of an individual, the system comprising:
a pressure signal determination module configured to determine a physiological pressure signal of the individual;
a wavelet transform module configured to transform the physiological pressure signal into a scalogram using at least one wavelet transform; and
a respiratory effort determination module configured to determine the respiratory effort of the individual through an analysis of the scalogram.

2. The system of claim 1, further comprising a pressure detection sub-system configured to directly detect a physiological pressure of the individual, wherein the pressure signal determination module determines the physiological pressure signal from the directly-detected physiological pressure of the individual.

3. The system of claim 2, wherein the pressure signal determination module is operatively connected to the pressure detection sub-system, and wherein the pressure signal determination module is configured to receive the physiological pressure from the pressure detection sub-system.

4. The system of claim 2, wherein the pressure detection sub-system comprises a pressure detection device including an arterial line (A-line) catheter configured to be implanted within vasculature of the individual, and wherein the A-line catheter directly detects the physiological pressure within the vasculature of the patient.

5. The system of claim 1, wherein the scalogram decouples the physiological pressure signal into multiple component parts, and wherein the respiratory effort determination module is configured to determine the respiratory effort of the individual through an analysis of one or more of the multiple component parts.

6. The system of claim 1, wherein the scalogram comprises one or more of a primary respiratory band, a secondary respiratory band, a primary pulse band, or a secondary pulse band, and wherein the respiratory effort determination module is configured to determine the respiratory effort of the individual through an analysis of one or more of the primary respiratory band, the second respiratory band, the primary pulse band, or the secondary pulse band.

7. The system of claim 1, wherein the respiratory effort determination module is configured to analyze the scalogram to determine a ridge amplitude defined by a signal amplitude multiplied by a constant.

8. The system of claim 7, wherein the constant is 0.67.

9. A method for determining respiratory effort of an individual, the method comprising:
determining a physiological pressure signal of the individual with a pressure signal determination module;
transforming the physiological pressure signal into a scalogram through at least one wavelet transform using a wavelet transform module; and
determining the respiratory effort of the individual through an analysis of the scalogram using a respiratory effort determination module.

10. The method of claim 9, further comprising directly detecting a physiological pressure of the individual with a pressure detection sub-system, wherein the determining operation comprises determining the physiological pressure signal from the directly-detected physiological pressure.

11. The method of claim 10, further comprising sending the physiological pressure from the pressure detection sub-system to the pressure signal determination module.

12. The method of claim 10, wherein the pressure detection sub-system comprises a pressure detection device including an arterial line (A-line) catheter configured to be implanted within vasculature of the individual, and wherein the method further comprises directly detecting the physiological pressure within the vasculature of the patient with the A-line catheter.

13. The method of claim 9, wherein the transforming operation comprises decoupling components parts of the physiological pressure signal in the scalogram, and wherein the determining operation comprises determining the respiratory effort of the individual through an analysis of one or more of the multiple component parts.

14. The method of claim 9, wherein the scalogram comprises one or more of a primary respiratory band, a secondary respiratory band, a primary pulse band, or a secondary pulse band, and wherein the determining operation comprises determining the respiratory effort of the individual through an analysis of one or more of the primary respiratory band, the second respiratory band, the primary pulse band, or the secondary pulse band.

15. The system of claim 9, wherein the determining operation comprises analyzing the scalogram to determine a ridge amplitude defined by a signal amplitude multiplied by a constant.

16. A tangible and non-transitory computer readable medium that includes one or more sets of instructions configured to direct a computer to:
determine a physiological pressure signal of the individual through a direct detection of the physiological pressure;
transform the physiological pressure signal into a scalogram through at least one wavelet transform; and
determine the respiratory effort of the individual through an analysis of the scalogram.

17. The tangible and non-transitory computer readable medium of claim 16, wherein the one or more instructions are further configured to direct the computer to decouple components parts of the physiological pressure signal in the scalogram, and determine the respiratory effort of the individual through an analysis of one or more of the multiple component parts.

18. The tangible and non-transitory computer readable medium of claim 16, wherein the one or more instructions are further configured to direct the computer to determine the respiratory effort of the individual through an analysis of one or more of the primary respiratory band, the second respiratory band, the primary pulse band, or the secondary pulse band of the scalogram.

19. The tangible and non-transitory computer readable medium of claim 16, wherein the one or more instructions are further configured to direct the computer to analyze the scalogram to determine a ridge amplitude defined by a signal amplitude multiplied by a constant.

20. The tangible and non-transitory computer readable medium of claim 16, wherein the constant is 0.67.
